(19) United States
(12) Patent Application Publication
(10) Pub. No.: US 2011/0087224 A1
(43) Pub. Date: Apr. 14, 2011

(54) MAGNETIC SURGICAL SLED WITH VARIABLE ARM

(76) Inventors: JEFFREY A. CADEDDU, DALLAS, TX (US); DANIEL J. SCOTT, DALLAS, TX (US); RAUL FERNANDEZ, ARLINGTON, TX (US); RUDOLPH H. NOBIS, MASON, OH (US); RICHARD A. BERGS, GRAND PRAIRIE, TX (US)

(21) Appl. No.: 12/789,245

(22) Filed: May 27, 2010

Related U.S. Application Data
(63) Continuation of application No. 12/576,529, filed on Oct. 9, 2009, now abandoned.

(51) Int. Cl. A61B 18/14; A61B 17/00

(52) U.S. Cl. 606/49; 606/1

(57) ABSTRACT

A surgical device comprises an ex vivo magnet and an in vivo sled magnetically attracted to the ex vivo magnet. The sled can be positioned and anchored within a patient by moving the ex vivo magnet. The sled defines a longitudinal axis. An arm extends from the sled. The arm being moveable relative the sled between a retracted position and an extended position. The arm comprises an end effector. A longitudinally oriented screw operatively is connected to the sled and arm such that rotation of the screw moves the arm between the retracted and extended positions.

### CROSS-REFERENCE TO RELATED APPLICATION

This is a continuation of co-pending application Ser. No. 12,576,529, filed Oct. 9, 2009, which is incorporated by reference without disclaimer.

### BACKGROUND

The present invention relates in general to surgical devices and procedures, and more particularly to minimally invasive surgery.

Surgical procedures are often used to treat and cure a wide range of diseases, conditions, and injuries. Surgery often requires access to internal tissue through open surgical procedures or minimally invasive surgical procedures. Minimally invasive surgery often involves using an endoscope, such as laparoscopes, arthroscopes, and flexible endoscopes, to visualize internal tissue of a patient, which sometimes referred to as “endoscopic surgery”. Endoscopes and instruments are typically introduced into a patient through percuateous punctures or incisions, or through a patient's natural orifices to access intraluminal anatomomy or for transluminal procedures.

Minimally invasive surgery has numerous advantages compared to traditional open surgical procedures, including reduced trauma, faster recovery, reduced risk of infection, and reduced scarring. Minimally invasive surgery is often performed with an insufflatory fluid present within the body cavity, such as carbon dioxide or saline, to provide adequate space to perform the intended surgical procedures. The insufflated cavity is generally under pressure and is sometimes referred to as being in a state of pneumoperitoneum. Surgical access devices are often used to facilitate surgical manipulation of internal tissue while maintaining pneumoperitoneum. For example, trocars may be used to provide a port through which endoscopes and surgical instruments are passed. Trocars generally have an instrument seal, which prevents the insufflatory fluid from escaping while an endoscope or surgical instrument is positioned in the trocar.

While a wide range of minimally invasive surgical devices and techniques have been used, one has previously made or used the devices and techniques in accordance with the present invention.

### BRIEF DESCRIPTION OF DRAWINGS

While the specification concludes with claims which particularly point out and distinctly claim the invention, it is believed the invention will be better understood from the following description taken in conjunction with the accompanying drawings illustrating some non-limiting examples of the invention. Unless otherwise indicated, like-numbered references refer to the same elements in the various figures. Unless otherwise indicated, the figures are not necessarily drawn to scale, but rather to illustrate the principles of the invention.

FIG. 1 depicts a cross-sectional view of a magnetically anchored surgical sled with an arm in a retracted position;

FIG. 2 depicts a cross-sectional view of a magnetically anchored surgical sled with an arm in an extended position;

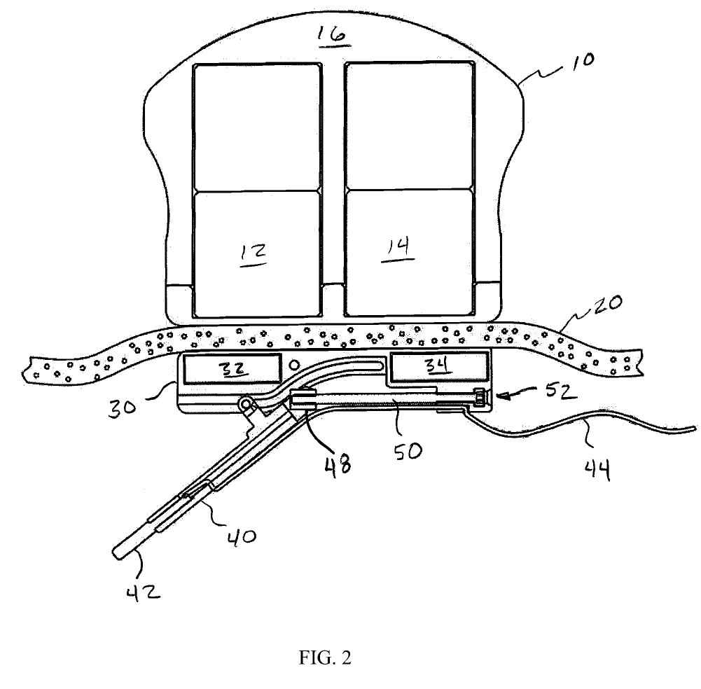

FIG. 3 depicts a isometric view of a magnetically anchored surgical sled with an arm in a retracted position;

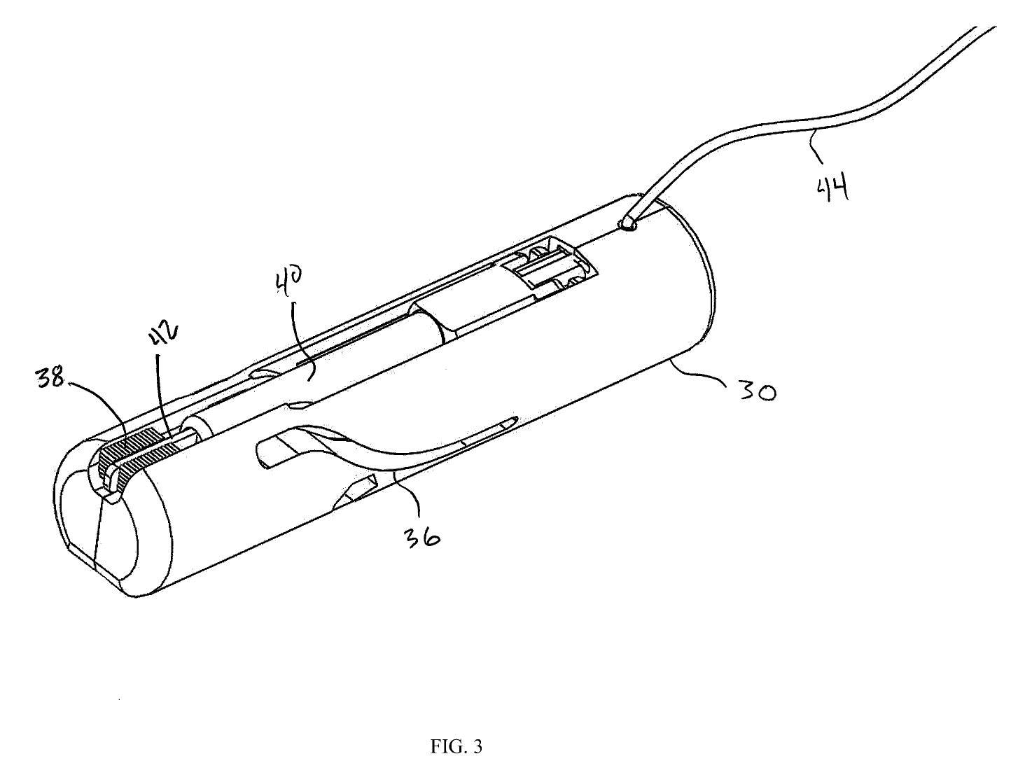

FIG. 4 depicts a isometric view of a magnetically anchored surgical sled with an arm in an extended position;

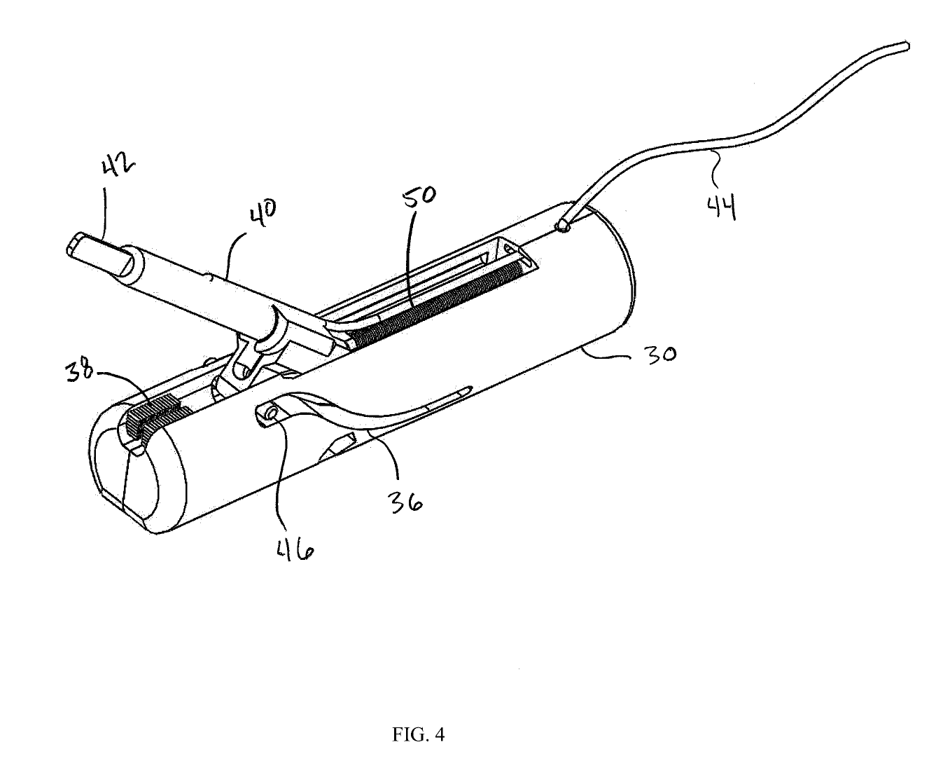

FIG. 5 depicts a isometric view of a magnetically anchored surgical sled with an arm in an extended position;

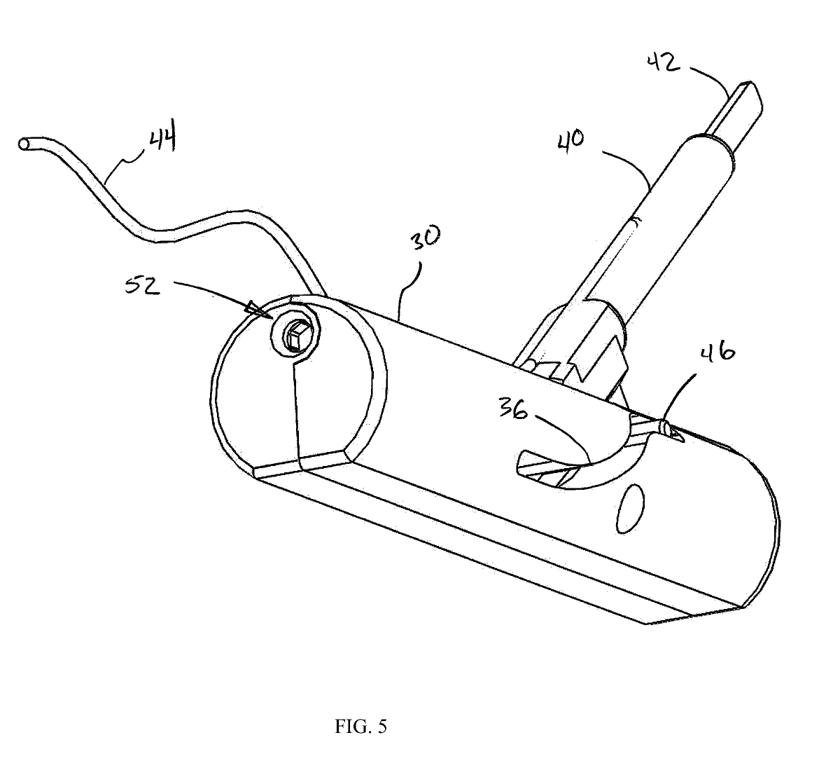

FIGS. 6A depicts a cross-sectional view of a magnetically anchored surgical sled with an arm in a retracted position;

FIGS. 6B depicts a cross-sectional view of a magnetically anchored surgical sled with an arm in a one quarter extended position;

FIGS. 6C depicts a cross-sectional view of a magnetically anchored surgical sled with an arm in a half extended position;

FIGS. 6D depicts a cross-sectional view of a magnetically anchored surgical sled with an arm in a three quarters extended position; and

FIGS. 6E depicts a cross-sectional view of a magnetically anchored surgical sled with an arm in a fully extended position.

### DETAILED DESCRIPTION

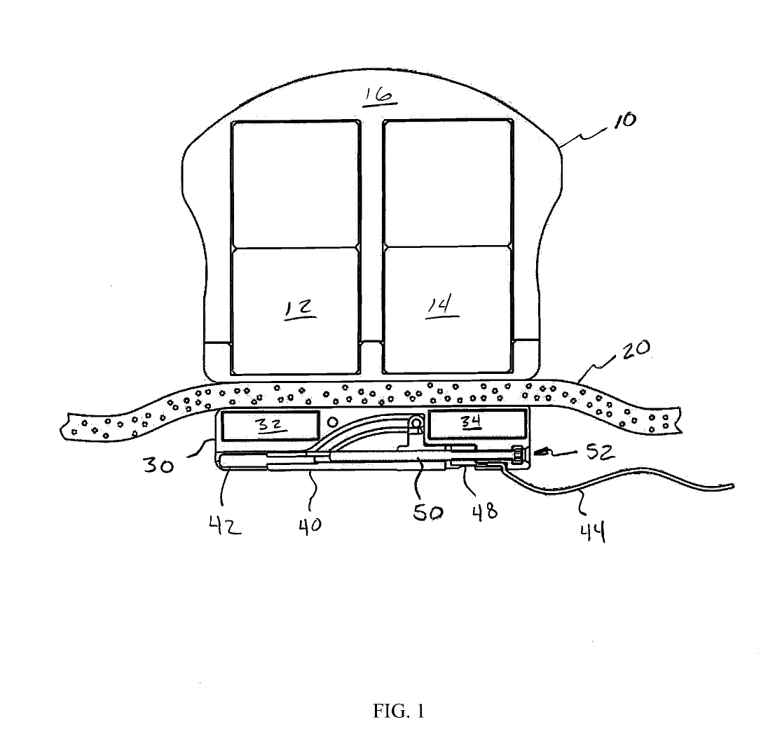

The embodiment shown in FIG. 1 comprises an anchor (10) and an sled (30). Patient tissue (20), such as the abdominal wall, an organ wall, or the like, is interposed between the anchor (10) and the sled (30). The anchor (10) and sled (30) and magnetically coupled to each other through the tissue (20). By sliding the anchor (10) relative the tissue (2), the surgeon can position the sled (30) in a desired location. Likewise, by keeping the anchor (10) stationary relative the tissue (20), the surgeon can anchor the sled (30) in a desired location. The anchor (10) will often be positioned ex vivo and the sled (30) positioned in vivo.

In the present embodiment, the anchor (10) includes two magnets (12, 14). The magnets (12, 14) are contained within a casing (16) that forms an ergonomic handle. The magnets (12, 14) can take a variety of forms such as permanent magnets, rare earth magnets, electromagnets, and the like. The magnets (12, 14) are magnetically coupled to supports (32, 34). The present embodiment the supports (32, 34) may be formed from a magnetic materials, such as any of a variety of known ferromagnetic materials or magnets. The pairing of two magnets (12, 14) with supports (32, 34), respectively, facilitate in vivo angular orientation of the sled (30). For instance, by rotating the anchor (10) relative the tissue (20), the sled (30) will likewise rotate.

The sled (30) can take a variety of different shapes and sizes; however, in the present embodiment the sled (30) is generally cylindrical in shape and sized to pass through a standard trocar, such as a 12 mm, 18 mm, or 20 mm trocar. The nominal length of the sled (30) may be between 60-90 mm, more preferably between 65-80 mm, and most preferably between 70-75 mm. The nominal diameter is the sled (30) may be between 12-19 mm, more preferably between 13-17 mm, and most preferably between 14-16 mm. The arm may be between 40 and 80 mm in length.

An arm (10) is connected to the sled (30). The arm (40) in the present example is substantially straight and rigid; however, curved, articulating, steerable arms, or flexible are also contemplated. The arm (40) includes an end effector (42), which in this example is a mono-polar electro-cautery tip. A variety of other end effectors could also be used, including graspers, scissors, ultrasonic blades, bi-polar clamps, surgical staplers, ultrasonic sensors, cameras, suturing devices, and the like. A tether (44) is operatively connected to the end effector (42) and extends from the sled (30). In the present example the tether (44) is a wire to deliver electrical energy to the electro-cautery tip; however, the type of tether (44) may depend upon the end effector (42). For instance, the tether could include push/pull wires to deliver forces, tubes to deliver fluids or pressure, fiber optic cables to deliver light or signals, electrical wires to deliver electricity or signals, and the like.

The arm (40) is moveable relative the sled (30) between a retracted position and an extended position. Preferably the arm (40) is at least partially recessed within the sled (30) when the arm (40) is in the retracted position. As shown in the present example, the arm (40) is completely recessed within the sled (30) in the retracted position. In the present embodiment an optional brush (38) is positioned on the sled (40) to interfere with the electro-cautery tip (42) when the arm is in the retracted position to clean the tip from any tissue residue or charring. In an alternative embodiment, a wiper blade made from an polymer or metal could also be used in place of or in conjunction with the brush (38).

A screw (50) is operatively connected to the sled (30) and arm (40) such that rotation of the screw (50) moves the arm (40) between the retracted and extended positions. In the present example the screw (50) is a leadscrew that is substantially oriented longitudinally with the sled (30). The screw (50) includes a head (52) accessible from one end the sled (30) to turn the screw (50). Any of a variety of head configurations may be used, including a hex ball head, hex head, flat head, phillips head, and the like. A nut (48) is connected to the arm (40) and threadedly engages the screw (50). The sled (30) includes a track (36) that receives a follower (46) connected to the arm (40). In the present embodiment the track (36) the track is non-linear. Also in the present embodiment, a portion of the track (36) runs substantially tangent with the longitudinal axis of the sled (30), and a portion of the track (36) runs transverse the longitudinal axis.

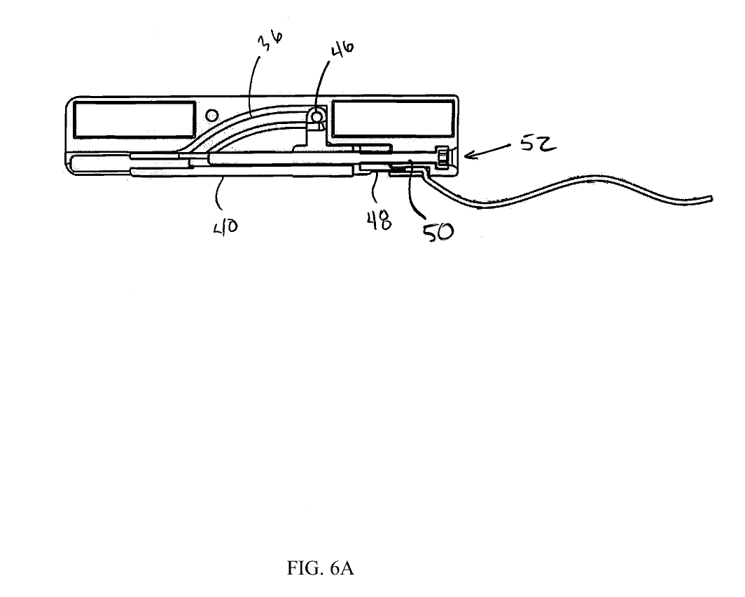

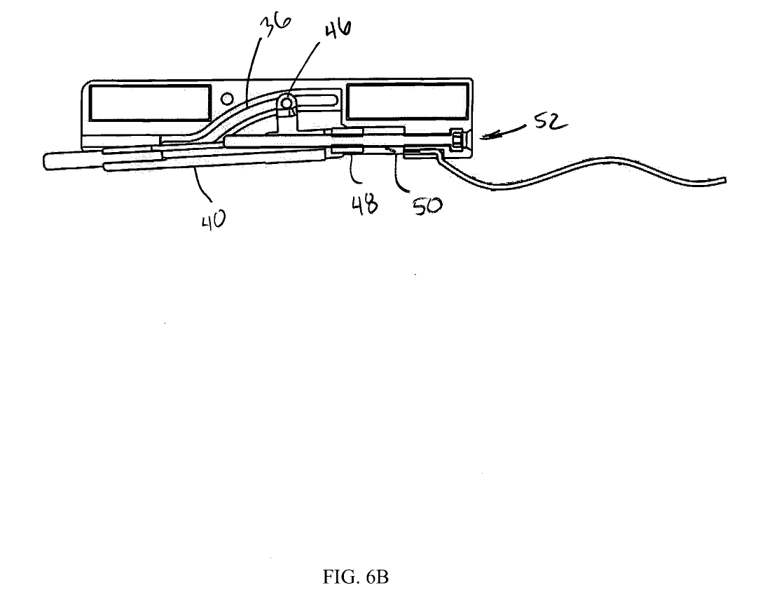

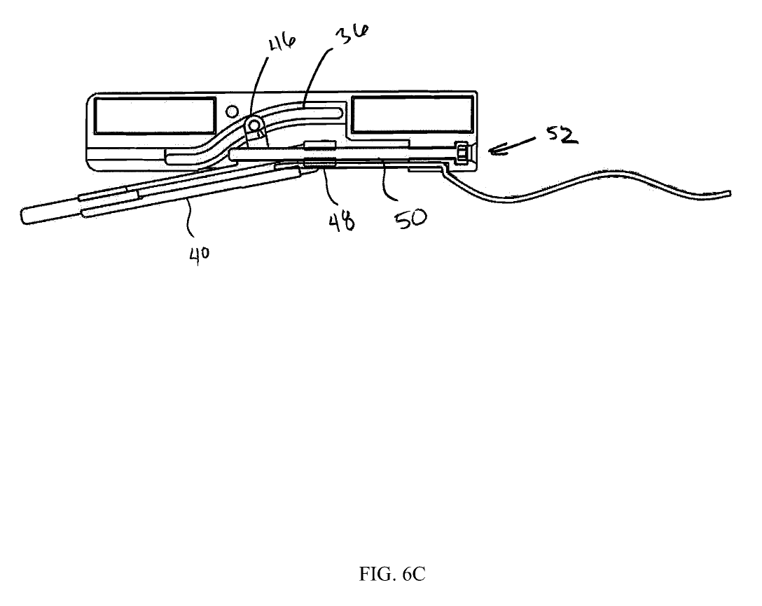

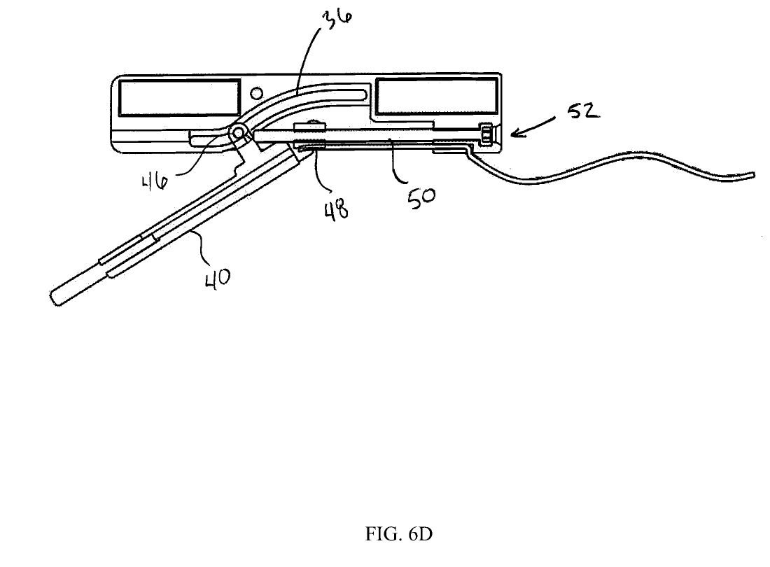

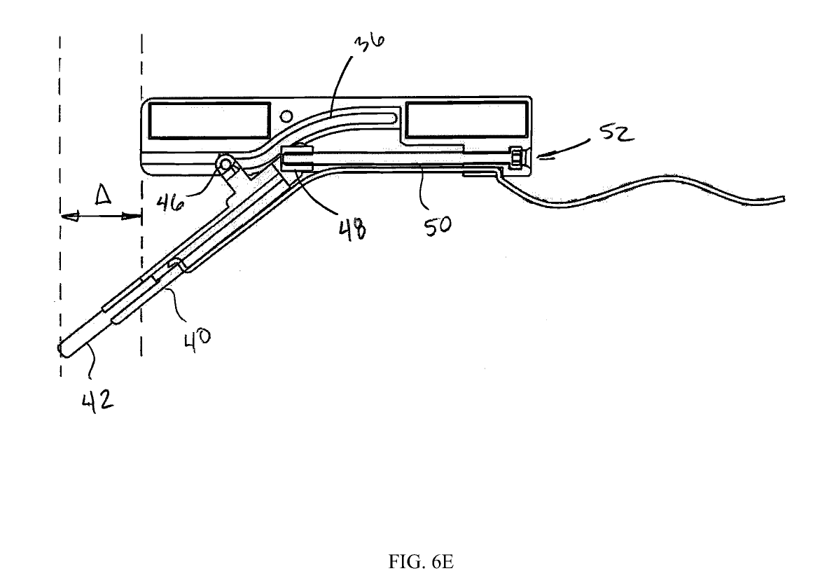

As illustrated in FIGS. 6A-E, rotation of the screw (48) is translated into longitudinal motion of the nut (48) thereby longitudinally translating the arm (30) relative the sled (30). As such, the follower (46) is advanced along the track (36) causing the arm to pivot laterally away from sled (30). Accordingly, the arm (40) both translates longitudinally and pivots laterally relative the sled (30). In the fully extended position as shown in FIG. 6E, the end effector (42) extends longitudinally beyond the sled (30) by the distance A, which may help facilitate accessing anatomical structures with the end effector (42). Another advantage of the present embodiment is that arm (40) will hold its position relative to the sled (30) anywhere between the fully retracted and fully extended positions. Therefore, the surgeon can select any position for the arm (40) best suited for the particular surgery and anatomy.

The following is one example of the device being used. The sled (30) is delivered into a patient's peritoneal cavity, preferably when the cavity is insufflated, with the arm (40) in the retracted position. The delivery may be through a percutaneous incision, such as through a trocar or other access device, or through a NOTES incision, such as transgastric, transvaginal, transcolonic, and the like. If the sled (30) includes a tether (44), it may be passed through the delivery incision or through a separate incision. The anchor (10) is placed ex vivo on the abdomen to attract and anchor the sled (30) to the abdominal wall. A slender rotational driver is passed into the peritoneal cavity through the same incision or a separate trocar. The rotational driver is dimensioned to mate with the head (52) and may be rigid or flexible. Usually under visualization from an endoscope, the driver is then positioned into the head (52) and rotated, either manually or with a motor, to extend the arm (40) to the desired position. The driver may then be removed from the surgical field. The surgeon will typically move and rotate the anchor (10) across the abdomen, and the sled (30) will follow due to the magnet attractions. By palpating and deflecting the abdomen with the anchor (10), either straight down or at an angle, the surgeon can move the end effector (42) to a desired location in the peritoneal cavity to perform a surgeon procedure. After completing the procedure, the driver may be reintroduced to the peritoneal cavity, into the head (52), and rotated in the opposite direction to retract the arm (40). The anchor (10) may then be removed from the abdomen, thus releasing the sled (30) from the abdominal wall. The sled (30) and driver may then be removed from the surgical field.

Having shown and described various embodiments and examples of the present invention, further adaptations of the methods and devices described herein can be accomplished by appropriate modifications by one of ordinary skill in the art without departing from the scope of the present invention. Several of such potential modifications have been mentioned, and others will be apparent to those skilled in the art. For instance, the specific materials, dimensions, and the scale of drawings will be understood to be non-limiting examples. Accordingly, the scope of the present invention should be considered in terms of the following claims and is understood not to be limited to the details of structure, materials, or acts shown and described in the specification and drawings.

1. A surgical device, comprising:
a) an ex vivo magnet;
b) an in vivo sled magnetically attracted to the ex vivo magnet, whereby the sled can be positioned and anchored within a patient by moving the ex vivo magnet, the sled defining a longitudinal axis;
c) an arm extending from the in vivo sled, the arm being moveable relative the sled between a retracted position and an extended position, the arm comprising an end effector; and
d) a longitudinally oriented screw operatively connected to the sled and arm such that rotation of the screw moves the arm between the retracted and extended positions.

2. The surgical device of claim 1, further comprising a track on the sled and a follower positioned in the track and connected to the arm.

3. The surgical device of claim 2, wherein at least a portion of the track runs in tangent with the longitudinal axis.

4. The surgical device of claim 2, where at least a portion of the track runs transverse the longitudinal axis.

5. The surgical device of claim 2, wherein the track is non-linear.

6. The surgical device of claim 1, wherein the end effector is an electro-cautery tip.

7. The surgical device of claim 6, further comprising a brush positioned on the sled, the brush interfering with the electro-cautery tip when the arm is in the retracted position.

8. The surgical device of claim 1, further comprising a tether operatively connected to the end effector and extending from the sled.

9. The surgical device of claim 1, wherein the arm is recessed within the sled in the retracted position.

10. The surgical device of claim 9, wherein the arm is completely recessed within the sled in the retracted position.

11. The surgical device of claim 1, wherein rotation of the screw longitudinally translates the arm relative the sled.

12. The surgical device of claim 11, wherein in the extended position the end effector extends longitudinally beyond the sled.

13. A surgical device, comprising:
a) an anchor;
b) a sled magnetically attracted to the anchor whereby the sled can be positioned within a patient by moving the anchor;
c) an arm extending from the sled, the arm being moveable relative the sled between a retracted position and an extended position, the arm comprising an end effector; and
d) a leadscrew operatively connected to the sled and arm such that rotation of the leadscrew moves the arm between the retracted and extended positions.

14. A surgical device, comprising:
a) an ex vivo magnet;
b) an in vivo sled magnetically attracted to the ex vivo magnet whereby the sled can be positioned within a patient by moving the ex vivo magnet;
c) an arm extending from the in vivo sled, the arm being moveable relative the sled between a retracted position and an extended position, the arm comprising an electro-cautery tip; and
d) a cleaning means on the sled, the cleaning means interfering with the electro-cautery tip when the arm is in the retracted position.
